SEQUENCE LISTING (6 sequences)

SEQ ID NO: 1 - DNA, 18 bp, Artificial Sequence
  Synthetic oligonucleotide primer
ccacgtgcgcagcaggac

SEQ ID NO: 2 - DNA, 20 bp, Artificial Sequence
  Synthetic oligonucleotide primer
ccgtcctgccccttcacctt

SEQ ID NO: 3 - DNA, 17 bp, Artificial Sequence
  Synthetic probe for the c.-124 C>T mutations in the promoter of the hTERT gene
  misc_feature (1)..(1): Yakima Yellow Dye
  misc_feature (17)..(17): TAMRA
agggcccggagggggct

SEQ ID NO: 4 - DNA, 17 bp, Artificial Sequence
  Synthetic probe for the c.-124 C>T mutations in the promoter of the hTERT gene
  misc_feature (1)..(1): FAM
  misc_feature (17)..(17): TAMRA
agggcccggaaggggct

SEQ ID NO: 5 - DNA, 18 bp, Artificial Sequence
  Synthetic probe for the c.-146 C>T mutations in the promoter of the hTERT gene
  misc_feature (1)..(1): Yakima Yellow Dye
  misc_feature (17)..(17): TAMRA
cggggacccgggaggggt

SEQ ID NO: 6 - DNA, 18 bp, Artificial Sequence
  Synthetic probe for the c.-146 C>T mutations in the promoter of the hTERT gene
  misc_feature (1)..(1): FAM
  misc_feature (17)..(17): TAMRA
cggggacccggaaggggt

(19) United States
(12) Patent Application Publication
(10) Pub. No.: US 2018/0155793 A1
(43) Pub. Date: Jun. 7, 2018

(54) METHOD, SEQUENCES, COMPOSITIONS AND KIT FOR DETECTION OF CHANGES IN THE PROMOTER OF THE GENE HTERT

(71) Applicant: IPATIMUP (INSTITUTO DE PATOLOGIA E IMUNOLOGIA MOLECULAR DA UNIVERSIDADE DO PORTO), Porto (PT)

(72) Inventors: Ana Paula SOARES DIAS FERREIRA, Porto (PT); Hugo Joäo MARQUES PRAZERES, Serpins (PT); Catarina Miguel ALVES SALGADO, Fanzeres (PT); Rui Pedro MONTEIRO BATISTA, Vila Real (PT); Joao Pedro RICO DE OLIVEIRA VINAGRE, Ovar (PT)

(73) Assignee: IPATIMUP (INSTITUTO DE PATOLOGIA E IMUNOLOGIA MOLECULAR DA UNIVERSIDADE DO PORTO), Porto (PT)

(21) Appl. No.: 15/569,964

(22) PCT Filed: May 2, 2016
(86) PCT No.: PCT/PT2016/050007

§ 371 Date: Oct. 27, 2017

(30) Foreign Application Priority Data
Apr. 30, 2015 (PT) 108419

(51) Int. Cl. C12Q 1/6886

(52) U.S. Cl. CPC C12Q 1/6886; C12Q 2600/156

(57) ABSTRACT

The present invention refers to a method for the detection of the c.-124 C>T and c.-146 C>T mutations in Htert gene promoter. The referred method uses a reaction composition that comprises primers for amplification and probes for genotyping.

Another aspect of this invention refers the primers and probes used in performing the aforementioned method with sequences, identified as SEQ ID nr.1 to SEQ ID nr.6, that display high specificity for these mutations, as well as compositions that contain them.

The present invention further refers to a kit comprising the above mentioned compositions for detecting mutations c.-124 C>T and c.-146 C>T mutations in Htert gene promoter by conducting the present method invention.

The method, gene sequences, compositions and kit of the present invention can be advantageously used for detecting trace amounts of c.-124 C>T and c.-146 C>T mutations, present in biological samples due to its high sensitivity and specificity for such mutations.

The present invention can therefore be applied in early detection, identification, detection of recurrence or prediction and monitoring of diseases associated with those mutations, such as bladder carcinomas, thyroid carcinomas, squamous cell carcinoma, basal cell carcinomas, skin cancer, central nervous system cancers and hepatocellular carcinoma, among others and eventually provide the basis for appropriate treatment setting.

Thus, the present invention falls within the technical field of medicine, pharmaceutics, molecular biology, biochemistry, and human related genetics.

### TECHNICAL FIELD OF THE INVENTION

The present invention refers to a method for the ultrasensitive detection of c.-124 C>T and c.-146 C>T mutations in Htert gene promoter, the reaction compositions comprising primers for amplification and probes for genotyping, the sequences of referred as SEQ ID nr.1 to SEQ ID nr.6, that were designed to display a high specificity for these mutations, as well as, the kit comprising such compositions for performing the referred method.

The present invention can advantageously be used to detect trace amounts of such c.-124C>T and c.-146C>T mutations present in biological samples and in vitro due to the high sensitivity of the method and specificity of gene sequences created, and allowing thereby the early detection, recurrence identification or prediction and monitoring of diseases associated with those mutations, such as bladder carcinomas, thyroid carcinomas, squamous cell carcinoma, basal cell carcinomas, skin cancer, central nervous system cancers and hepatocellular carcinoma, among others and eventually provide the basis for appropriate treatment setting. Thus, the present invention falls within the technical field of medicine, pharmaceutics, molecular biology, biochemistry, and human related genetics.

### STATE OF THE ART

Somatic mutations that occur during DNA replication that precede a mitotic division, are at the origin of certain human disorders, particularly certain types of cancers. The presence of the enzyme telomerase was observed in a high percentage, about 75-80%, of tumor cell lines. Recently, the applicants have identified mutations in the telomerase gene promoter encoding this enzyme, the TERT gene, particularly, the mutations at positions c.-124C>T and c.-146C>T (from the initial ATG). These mutations have been associated with several malignancies, such as central nervous system cancers (43-51%), bladder carcinomas (59-66%), hepatocellular carcinomas (59%), thyroid carcinomas (10%), skin cancers (melanoma 29%-73%, 73% basal cell carcinoma (BCC) and squamous cell carcinomas (SCC) 45%) and gastrointestinal stromal tumours (GISTs) (4%) [1-7].

The fact that mutations in telomerase promoter are present in a relatively high frequency in certain cancers makes them potential biomarkers for early detection, diagnosis, prognosis, recurrence detection or prediction/monitoring of therapy response of tumors, such as, central nervous system tumours, bladder carcinomas, hepatocellular carcinomas, thyroid carcinomas, and skin cancers, among others. For example, in bladder cancer, somatic mutations in positions c.-124C>T and c.-146C>T (from the ATG) of the telomerase gene have a prevalence that reaches 85%, and, along with the mutations R248C and S249C in the fibroblast growth receptor 3 (FGFR3) gene [9], are the most reliable biomarkers for bladder cancer.

However, for the in vitro use of biological sample fluids (e.g. urine, plasma, cerebrospinal fluid, aspiration cytology, among others), the mutation detection method in positions c.-124 C>T and c.-146C>T (from the ATG) of the telomerase gene must have high analytical sensitivity in order to detect trace amounts of the mutated alleles, even when they are “diluted” in a sample with greater abundance of alleles “wild-type”.

The document WO2015153808 discloses methods and compositions useful in the detection of some diseases, in particular cancer, more specifically, thyroid cancer, related to mutations at positions c.-124C>T and c.-146C>T of the TERT gene. However, the nucleotide sequences disclosed in that document are used for detection of such mutations by direct amplification methods, sequence dependent, and have limited sensitivity when the presence of normal sequences is relatively high compared to the mutant sequences. On the other hand, they are susceptible to the occurrence of non-specific amplification, so that, they do not exhibit high specificity for this type of mutation, which causes, in its general method not to be sufficiently sensitive and specific.

The document WO2014160834 discloses methods and compositions useful in the detection and treatment of different types of thyroid cancer, related to mutations at positions c.-124 C>T and c.-146 C>T TERT gene. However, in this document the disclosed nucleotide sequences did not show high sensitivity since the presence of normal sequences is very abundant when compared to the mutant sequences, and the proposed method is based on DNA sequencing using “BigDye terminator v3.1” sequencing ready kit and in a Applied Biosystems ABI PRISM equipment 3730, which has a limited sensitivity to detect rare alleles in the reaction. Moreover, the presence of the mutation must be confirmed by sequencing in both directions, sense and antisense, which makes the method impractical and laborious and apply only to thyroid cancers.

Thus, it is necessary to develop a method with sufficient sensitivity and specificity that allows the detection of such mutations in biological samples even in conditions in which the mutated alleles are present in very low amounts.

The present invention proposes an ultrasensitive method that allows the detection of mutations c.-124 C>T and c.-146 C>T in Htert gene promoter, by designing primers and probes whose sequences are specific for this purpose.

### SUMMARY OF THE INVENTION

The present invention refers to a primer set and probe sequences (SEQ ID SEQ ID nr.6 nr.1), for DNA amplification and hybridization of biological samples, which were designed to present high specificity in detection of mutations c.-124 C>T and c.-146 C>T in Htert gene promoter according to claim 1.

These primers and DNA probes, because they have high specificity in the detection of such mutations allow its use in a method for high sensitivity detection of these mutations.

The present invention also refers to compositions for DNA amplification and hybridization, using techniques of PCR (Polymerase Chain Reaction) comprising the referred sequences of primers and DNA probes according to claim 2.

These compositions are applicable to the detection of mutations c.-124 C>T and c.-146 C>T in Htert gene promoter, that may be pre-prepared or may be prepared at the time of performing the detection method of the invention.

Another aspect of this invention refers to compositions for detecting mutations c.-124 C>T and c.-146 C>T in Htert gene promoter comprising the compositions of the PCR plus the human DNA from a biological sample to be tested for this mutation according to claim 7.

In these compositions, the amount of DNA in the test sample can be quite low, given the high specificity of the primers and probes of the present invention designed for that purpose.

Another aspect of the present invention relates to a kit comprising the primer composition and specific genetic probes to detect mutations c.-124 C>T and c.-146 C>T in Htert gene promoter, pre-prepared according to claim 9.

This kit has the advantage of developing ultrasensitive method of in vitro detection of mutations c.-124 C>T and c.-146 C>T in Htert gene promoter, a quick and reliable manner, by adding a sample human DNA test for the detection of said mutation.

Another aspect of this invention refers to the method of detection in vitro for mutations c.-124 C>T and c.-146 C>T in Htert gene promoter, using reaction compositions comprising primers for amplification and probes for genotyping, whose sequences are specific to these mutations, according to claim 11.

This method may advantageously be used to detect trace quantities of such mutations c.-124 C>T and c.-146 C>T present in biological samples due to its high sensitivity conferred by the genetic sequence designed for this purpose and which have a high specificity for such mutations.

Thus, the present invention can be applied in early detection, identification, recurrence or prediction detection and monitoring of diseases associated with such mutations, such as bladder carcinomas, thyroid carcinomas, skin cancers, central nervous system cancers and hepatocellular carcinoma among other and eventually provide the basis for the appropriate treatment setting.

Thus, another aspect of the present invention relates to a method of treatment where a suitable amount of a Htert gene promoter inhibitor is used in an individual that tests positive in the mutation detection test for c.-124 C>T or c.-146 C>T in the Htert gene promoter of the present invention according to claim 15.

### DESCRIPTION OF THE INVENTION

The present invention refers to a method for in vitro detection of mutations c.-124 C>T and c.-146 C>T M in Htert gene promoter. The referred reaction method uses compositions comprising primers for amplification and probes for genotyping, whose sequences (SEQ ID nr.1 to SEQ ID nr.6) are specific for these mutations.

Thus, primer sequences are created for amplification and probes for genotyping in order to build the basis for said amplification reaction, eg. a PCR assay Real-Time (RT-PCR) or Digital Time PCR. 6 sequences were designed for the primers and probes, as shown in the sequence list. This includes the sequences for the following purposes:
a. Detection of the mutation located at -124 bases upstream of the ATG of the hTERT gene
   SEQ ID n.1: Initiator F
   SEQ ID n.2: Initiator R
   SEQ ID n.3: Probe—124C
   SEQ ID n.4: Probe—124T
b. Detection of the mutation located at -146 bases upstream of the ATG of the hTERT gene
   SEQ ID n.1: Initiator F
   SEQ ID n.2: Initiator R
   SEQ ID n.3: Probe—146C
   SEQ ID n.4: Probe—146T

The above sequences can be prepared by the nucleotide synthesis method using the solid phase phosphoramidite nucleosides as described in the publication: Beaucage, S L; Iyer, R P (1992). “Advances in the Synthesis of Oligonucleotides by the Phosphoramidite Approach”. Tetrahedron 48 (12): 2223. The synthesis can be performed on columns with solid support (controlled pore glass or polystyrene) functionalized with the first base of the 3′ end of each oligonucleotide. The preparation of each oligonucleotide follows after a number of synthesis cycles, each consisting of chemical reactions, typically four chemical reactions: i) release (detritylation), ii) coupling, iii) protection and iv) oxidation. In each cycle are added at the 5′ terminus of the growing chain, nucleotide residues corresponding to the desired sequence.

These can be further modified at the 5′ and 3′ sequences of the probes by the addition of corresponded fluorophores such as FAM, Yakima Yellow, quencher (TAMRA), etc. known to experts, for the purpose of amplification and hybridization reactions in multiplex PCR. Thus, suitable fluorophores in the present invention are, for example compounds known to Alexa Fluor FAM, TET, JOE, VIC, HEX, Cy3, ATTO 550, TAMPA, ROX, Cy5, Cy5.5.

The nucleotides refer to nucleotides of natural or synthetic origin, with the hybridization capacity by base-pairing with complementary nucleotides, and may include, without limitation, DNA, RNA, and nucleotide analogues (e.g. nucleic acids with closed conformation, known as “locked nucleic acids”—LNA) nucleotides or without inter-nucleotide phosphodiester type linkages (e.g. peptide nucleic acid—PNA) and nucleic acids with tiodiester bonds, or the like for the same purpose.

Compositions for amplification and DNA hybridization (PCR compositions) were prepared by adding reactant solutions to PCR, available on the market, with different nucleotide sequences with SEQ ID Nr.1 SEQ ID nr.6 in different concentrations, and bi-distilled water and deionized water-bi.

The reaction solutions for PCR using vary with the desired type of amplification reaction. Examples of suitable reactant solutions for PCR for the embodiment of the present invention are for example the solution “TaqMan® Universal PCR Master Mix” Thermofischer Scientific company or similar solutions for the purpose of DNA amplification and hybridization.

The concentrations of probes refer to the molar concentration of the solution, corresponding to the number of moles of each probe per volume of solution, wherein nanoMolar (nM) corresponds to 1×10⁻⁹ mole per liter.

Compositions for PCR were prepared comprising various combinations of different concentrations of the probes with sequences SEQ ID Nr.3 SEQ ID nr.6. As mentioned previously, probes whose sequences are defined by SEQ ID Nr.3 and SEQ ID nr.4 were designed for the specific detection of c.-124 C>T mutation in the Htert gene promoter while the probes SEQ ID nr.5 and SEQ ID nr.6 were designed for the specific detection of c.-146 C>T mutation in the Htert gene promoter.

Thus, the compositions of the invention for PCR may comprise only one or two sets of probes mentioned above, i.e., the compositions of the PCR of the invention can comprise only the probes with SEQ ID NO. 3 and SEQ ID nr.4 (c.-124 set), the probes with the probes with SEQ ID nr.5 and SEQ ID nr.6 (c.-146 set), or with all the probes with SEQ ID Nr.3 SEQ ID nr.6 (c.-124+c.-146 complete set).

Where the compositions of the invention comprise just one probe set, set c.-124 or c.-146 together, then they are specific only for the detection of the respective mutated c.-124 C>T or C.-146 C>T. In the event that both sets of probes are present in the compositions for PCR, then these compositions are specific for the detection of both c.-124 C>T and c.-146 C>T mutations.

The concentration of nucleotide probes of the present invention in the solutions is adjusted to the PCR, in order to promote the sensitivity of the method of the invention the detection of such mutations. Preferably, the final concentration of each probe is adjusted to values between 400 and 1600 nM, more preferably with a final concentration values of 800 to 1600 nM and in a further preferred embodiment of the invention the values of the final concentration of each probe is approximately 1600 nM.

Thus, it is possible to obtain compositions for PCR comprising different combinations of final concentration values of each of the probes defined by SEQ ID Nr.3 and SEQ ID nr.6.

Thus, in a preferred form, the compositions for PCR comprising the following concentrations of the probes with SEQ ID Nr.3 SEQ ID nr.6:
i. of 250 nM to 500 nM of SEQ ID Nr.3, 400 nm to 1600 nM of SEQ ID nr.4 of 250 nM to 500 nM of SEQ ID nr.5 and 400 nm to 1600 nM of SEQ ID nr.6,

or
ii. of 250 nM to 500 nM of SEQ ID Nr.3, of 800 nm to 1600 nM of SEQ ID nr.4 of 250 nM to 500 nM of SEQ ID nr.5 and 800 nm to 1600 nM of SEQ ID nr.6.

The compositions of the reaction mixtures for the detection of mutations c.-124 C>T and c.-146 C>T in the Htert gene promoter were prepared by adding DNA from the biological sample to be tested to the compositions mentioned above for PCR.

The test DNA can be obtained from tissue, urine, circulating tumour DNA, germline or other biological sources using standard methods known in the art such as those commercialized by Qiagen QIAamp® company or similar methods. The amount of DNA to be tested can be present in the composition to detect mutations c.-124 C>T and c.-146 C>T in Htert gene promoter, at concentrations below 500 ng, less than 100 ng, 50 ng and the upper to 1 ng.

The referred adjust in the concentration of the probes used is intended to boost the analytical sensitivity of the method of the present invention, demonstrated by a minimum number of mutant alleles (DNA MUT) detected relative to the number of alleles “wild-type” (DNA WT). It is assumed that the increase in the analytical sensitivity results from increased concentrations of the probes used in the reaction by favouring amplification of the mutant allele.

Simultaneously, it reduced the efficacy of hybridization and amplification of the allele “wild-type” in order to keep as much as possible the balance of the reaction in favour of amplification of the mutant allele.

Thus, it was possible to develop a method in which various combinations of different concentrations of the probes used give rise to conditions that boost the analytical sensitivity and can detect very low levels of mutant alleles.

Up to certain levels, the increase in the concentration of mutated probe does not result in nonspecific detection of allele “wild-type” as is evidenced by the absence of signal from probes -124T and -146T when using only DNA WT-like sample (FIG. 5—panels C and D). Thus, based on the manipulation of the probe concentrations used, it is possible to calibrate the sensitivity of the analytical method while its specificity is retained.

In reflexion of this increased efficiency in the detection of mutated alleles -124T and -146T, is the significantly increase of the analytical sensitivity of the method, passing to minimum detection values of 1.56% for -124T allele and 0.78% for -146T allele (FIGS. 6A and B, respectively).

Thus, the method of the present invention comprises the steps of: i) preparing a composition for detecting mutations c.-124 C>T and/or c.-146 C>T M in Htert gene promoter, such as described above, ii) promotion of DNA extracted from a biological sample to be tested for the mutation, a composition for PCR amplification and hybridization, also as described above, iii) amplification and hybridization of the sample composition DNA for detecting mutations c.-124 C>T and/or c.-146 C>T in the Htert gene promoter by PCR reaction, real time PCR, RT-PCR or digital PCR or even multiplex PCR reaction, and iv) analysing the amplification curves thus obtained.

The presence of c.-124 mutated C>T and/or c.-146 C>T in the Htert gene promoter of test sample is analysed for the existence of an exponential growth of a fluorescence signal, which corresponds to the presence of at least one of the referred mutations.

The method of the present invention is useful in detection of trace amounts of c.-124 C>T and c.-146 C>T mutations in several biological fluids, e.g., urine, plasma, cerebrospinal fluid, aspiration cytology, among others, having as input obtained DNA from these different sources. In these examples, the advantage is to be able to make a determination from a less invasive kind of sample without having to rely on biopsy or surgery.

It can be applied in the surveillance of patients with bladder cancer via a DNA-based analysis result of a sample of urine. This application is based the fact that the mutation c.-124C>T and c.-146C>T are highly frequent in bladder cancer and can be detected in DNA derived from urine (FIG. 7). For patients who are in clinical surveillance because they have 50-70% chance of recurrence, the possibility of detecting recurrence for urine testing has advantages over the conventional method based on cystoscopy to be completely non-invasive and more convenient for the patient.

The method of the present invention may also be applied to the detection of other types of cancer in which the Htert gene mutations are present and for other clinical purposes. One example is in the fine needle aspirates analysis of thyroid nodules in order to provide prognostic information useful in deciding the need for an ablative treatment or lymphadenectomy extension.

Another example of applying the present invention relates to early detection, diagnosis, prognosis, recurrence detection or prediction/monitoring response to therapy of tumors such as central nervous system cancers, bladder carcinomas, hepatocellular carcinomas, thyroid carcinomas, skin cancers, among others.

The present invention may also be useful for tumor tissue analysis or circulating DNA in the context of “liquid biopsy” for possible selection and treatment of candidate patients, response and prediction to treatment and monitoring, for example, with telomerase inhibitors for the Htert gene promoter.

### BRIEF DESCRIPTION OF THE FIGURES

FIG. 1: Demonstration of the ability of the assay to detect mutations in the Htert gene promoter (TERTp) c.-124 C>T.

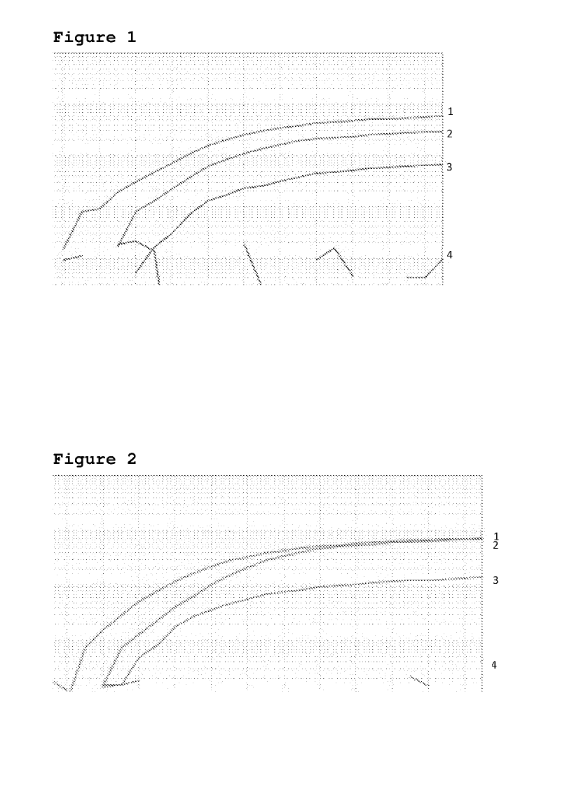

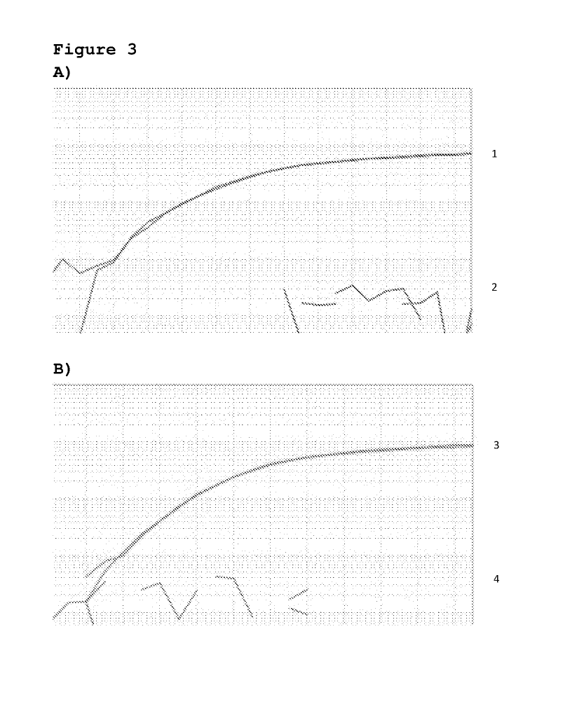

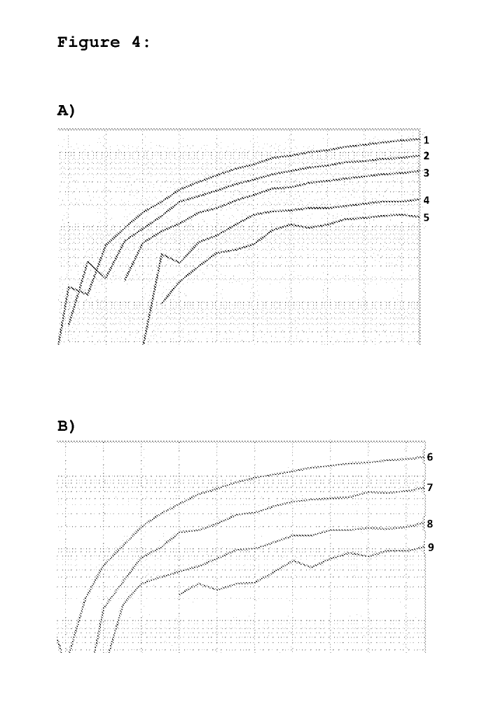

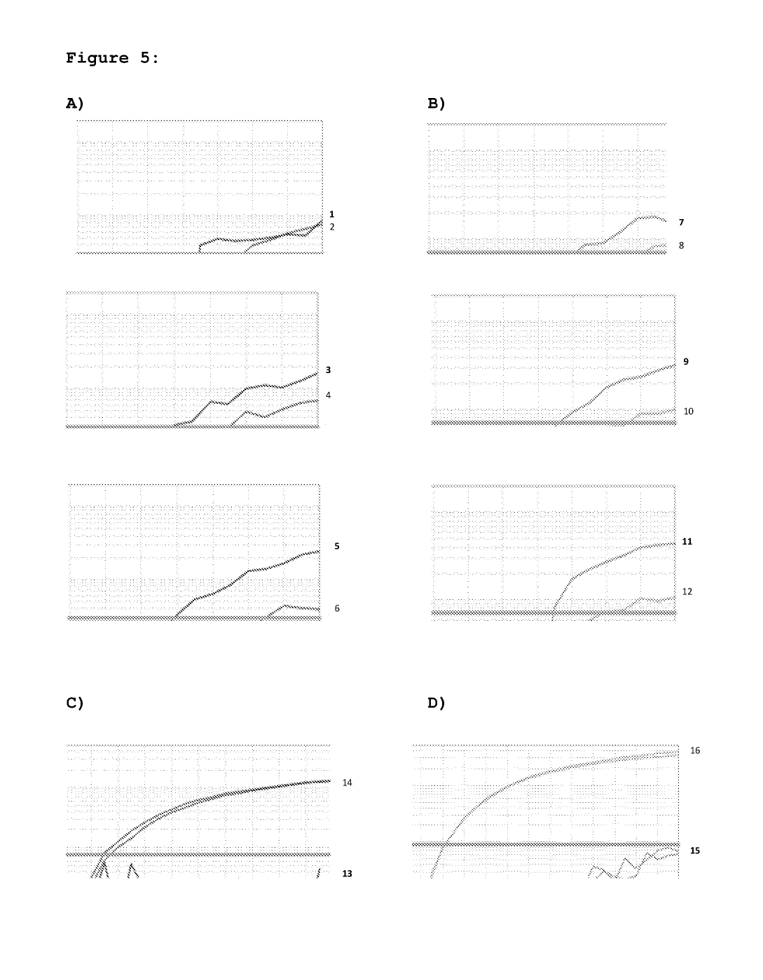

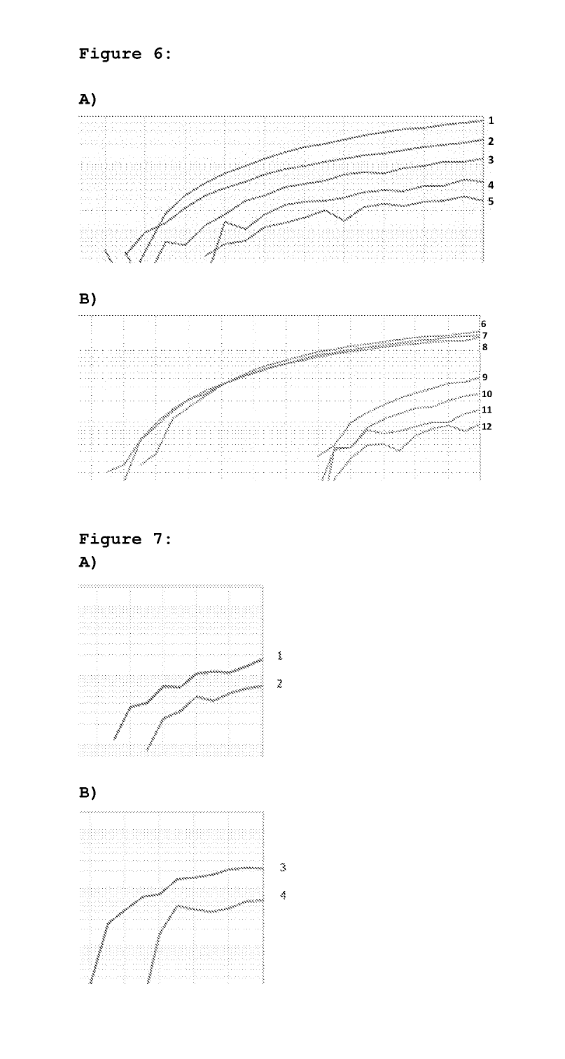

In this figure it is presented that the assay is specific for the detection of alleles at position -124. The performance of the assay is confirmed by testing the c.-124 C>T mutation detection in heterozygous DNA samples for c.-124 C>T mutation (DNA HET124) and cases “wild-type” (DNA WT). The results demonstrate that the probe specific for the mutant allele (−124 Probe T) generates a signal in HET-124 DNA sample and not the WT DNA sample and the probe for allele “wild-type” (Probe-124 C) generates a signal amplification in both samples.

1—amplification curve obtained with the probe -124C (allele “wild-type”) in DNA WT.

2—amplification curve obtained with the probe -124C (allele “wild-type”) in HET-124 DNA.

3—amplification curve obtained with -124T probe (mutant allele) in HET-124 DNA.

4—amplification curve obtained with -124T probe (mutant allele) in WT DNA.

X axis—amplification cycle

Y axis—Fluorescence change

FIG. 2: Demonstration of the ability of the assay to detect mutations in TERTp c.-146 C>T

In this figure it is presented that the assay is specific for the detection of alleles at position -146. The performance of the test in detecting the mutation c.-146 C>T is confirmed by the analysis of heterozygous DNA samples for c.-146 mutated C>T (DNA HET146) and cases wild-type (WT DNA). The results show that the probe specific for the mutant allele (−146 Probe T) generates a signal in HET-146 DNA sample and not in the WT DNA sample and the probe for allele “wild-type” (Probe-146 C) generates a signal amplification in both samples.

1—amplification curve obtained with the probe -146C (allele “wild-type”) in DNA WT.

2—amplification curve obtained with -146T probe (mutant allele) in HET-146 DNA.

3—amplification curve obtained with the probe -146C (allele “wild-type”) in HET-146 DNA.

4—amplification curve obtained with -146T probe (mutant allele) in WT DNA.

X axis—amplification cycle

Y axis—Fluorescence Change

FIG. 3: Evaluation of the analytical specificity of the tests:

(A) specific detection of mutation TERTp c.-124 C>T

(B) specific detection of mutation TERTp c.-146 C>T

In this figure it is demonstrated that the assay does not have cross-reactivity between the tested mutations.

FIG. 3-A: DNA sample heterozygous for the mutation TERTp-146C>T is not amplified by the probe -124T and a single probe to emit fluorescence is in fact the probe -124C (corresponding to allele “wild-type” at position -124)

FIG. 3-B: On the other hand, a DNA sample heterozygous for the mutation TERTp-124C>T is not amplified by the probe -146T and a single probe to emit fluorescence is in fact the probe -146C (corresponding to allele “Wild-Type” at position -146).

1—amplification curve obtained with the probe -124C (allele “wild-type”) in HET-146 DNA.

2—amplification curve obtained with -124T probe (mutant allele) in HET-146 DNA.

3—amplification curve obtained with the probe -146C (allele “wild-type”) in HET-124 DNA.

4—amplification curve obtained with -146T probe (mutant allele) in HET-124 DNA.

X axis—amplification cycle

Y axis—Fluorescence Change

FIG. 4: Evaluation of analytical sensitivity for detection of the -124T allele (A) and -146T (B) under conditions that mimic DNA samples derived from biological fluids, in which there are small amounts of mutated alleles in a global context where the predominant allele is the “wild-type”. DNA samples were used with c.-124C>T mutation or samples with c.-146C>T mutation serially diluted in “wild-type” DNA samples.

This figure reports the efficiency and sensitivity of the test in detecting mutated alleles -124T and -146T.

1—amplification curve obtained with probe -124T at 50% DNA MUT124/DNAWT.

2—amplification curve obtained with the probe -124T 25% DNA MUT124/DNAWT.

3—amplification curve obtained with the probe -124T 12.5% DNA MUT124/DNAWT.

4—amplification curve obtained with -124T probe at 6.25% DNA MUT124/DNAWT.

5—amplification curve obtained with -124T probe 3.125% DNA MUT124/DNAWT.

6—Amplification curve obtained with probe -146T at 50% DNA MUT146/DNAWT.

7—amplification curve obtained with the probe -146T 25% DNA MUT146/DNAWT.

8—amplification curve obtained with the probe -146T 12.5% DNA MUT146/DNAWT.

9—amplification curve obtained with -146T probe at 6.25% DNA MUT146/DNAWT.

X axis—amplification cycle

Y axis—Fluorescence Change

FIG. 5: Potentiation of the analytical sensitivity in detecting -124T allele (A) and -146T (B).

DNA samples were used with c.-124C>T mutation or samples with c.-146C>T mutation serially diluted in “wild-type” DNA samples in a proportion of 25% MUT DNA/DNA-WT.

It can be observed that in a low concentration condition of the probe for allele “wild-type” (e.g. 250 nM) the use of a higher concentration of the probe for the mutated alleles -124T and -146T (400-800-1600 nM) results in a significant increase of the detection signal of the mutated alleles (FIG. 5 panels A and B, respectively).

In panels C and D of this figure it can be seen that up to certain levels, the increase in the concentration of mutated probe does not result in nonspecific detection of allele “wild-type” as is evidenced by the absence of signal from probes -124T and -146T when using only DNA-WT as a sample.

1—amplification curve -124T probe (mutant) at a concentration of 400 nm.

2—amplification curve -124C probe (wild-type) at a concentration of 250 nM.

3—amplification curve -124T probe (mutant) at a concentration of 800 nm.

4—amplification curve -124C probe (wild-type) at a concentration of 250 nM.

5—amplification curve -124T probe (mutant) at a concentration of 1600 nM.

6—Amplification curve -124C probe (wild-type) at a concentration of 250 nM.

7—amplification curve -146T probe (mutant) at a concentration of 400 nm.

8—Amplification curve -146C probe (wild-type) at a concentration of 250 nM.

9—Amplification curve -146T probe (mutant) at a concentration of 800 nm.

10—amplification curve -146C probe (wild-type) at a concentration of 250 nM.

11—amplification curve -146T probe (mutant) at a concentration of 1600 nM.

12—amplification curve -146C probe (“wild-type”) at a concentration of 250 nM.

13—amplification curve -124T probe (mutant) at a concentration of 1600 nM.

14—amplification curve -124C probe (“wild-type”) at a concentration of 250 nM.

15—amplification curve -1246T probe (mutant) at a concentration of 400 nm.

16—amplification curve -146C probe (“wild-type”) at a concentration of 250 nM.

X axis—amplification cycle

Y axis—Fluorescence Change

FIG. 6: Evaluation of analytical sensitivity for detection of the -124T allele (A) and -146T (B) when increasing the concentration of the probe to the mutant allele and lowering the probe concentration used for allele “wild-type”. This change in the relative composition of the probes results in increased analytical sensitivity.

This figure presents the increased efficiency and sensitivity in the detection of mutated alleles -124T and -146T: the minimum detection values became 1.56% for -124T allele and 0.78% for -146T allele).

1—amplification curve obtained with the probe -124T 25% DNA MUT124/DNAWT.

2—amplification curve obtained with the probe -124T 12.5% DNA MUT124/DNAWT.

3—amplification curve obtained with -124T probe at 6.25% DNA MUT124/DNAWT.

4—amplification curve obtained with -124T probe 3.125% DNA MUT124/DNAWT.

5—amplification curve obtained with the probe -124T 1.56% DNA MUT124/DNAWT.

6—Amplification curve obtained with probe -146T at 50% DNA MUT146/DNAWT.

7—amplification curve obtained with the probe -146T 25% DNA MUT146/DNAWT.

8—amplification curve obtained with the probe -146T 12.5% DNA MUT146/DNAWT.

9—amplification curve obtained with -146T probe at 6.25% DNA MUT146/DNAWT.

10—amplification curve obtained with -146T probe 3.125% DNA MUT146/DNAWT.

11—amplification curve obtained with the probe -146T 1.56% DNA MUT124/DNAWT.

12—amplification curve obtained with the probe -146T 0.78% DNA MUT146/DNAWT.

X axis—amplification cycle

Y axis—Fluorescence Change

FIG. 7: Demonstration of the assay ability to detect TERTp mutations c.-124 C>T and c.-146 C>T in urine samples of patients with bladder cancer. In this figure you can be seen detection of c.-124 C>T mutation or c.-146 C>T mutation in urine samples. The results show that this type samples obtained from patients with bladder cancer, mutated alleles are detected for mutation -124 (A) or the -146 mutation (B) as well as signals to the corresponding alleles “wild-type”.

1—amplification curve obtained with probe -124C (allele “wild-type”) in DNA obtained from a urine sample of a patient with bladder cancer.

2—amplification curve obtained with -124T probe (mutant allele) in DNA obtained from a urine sample of a patient with bladder cancer.

3—amplification curve obtained with probe -146C (allele “wild-type”) in DNA obtained from a urine sample of a patient with bladder cancer.

4—Amplification -146T curve obtained with probe (mutant allele) in DNA obtained from a urine sample of a patient with bladder cancer.

X axis—amplification cycle

Y axis—Fluorescence Change

### EXAMPLES

### Example 1—Preparation of the Sequences of Primers and Probes

Two primer sequences were prepared, SEQ ID nr.1 and SEQ ID Nr.2, and 4 other sequences of the probes, SEQ ID Nr.3 SEQ ID nr.6 as follows:

The sequences indicated above were prepared by nucleotide synthesis method using the solid phase phosphoramidite nucleoside. The synthesis was performed in packed columns with the functionalized solid support with the first base of the 3′ end of each oligonucleotide. The preparation of each oligonucleotide followed after a number of synthesis cycles, each consisting of four chemical reactions:

Unlock (detritylation); coupling; protection and oxidation. In each cycle were added step by step, the 5′ terminus of the growing chain, nucleotide residues corresponding to the desired sequence. The concentration of probes was adjusted to values of 400 to 1600 nM by dilution of a lyophilized preparation in double distilled water and double deionized water.

### Example 2—Preparation of Compositions PCR Reaction

Several PCR reactions compositions were prepared comprising the nucleotide according to SEQ ID sequences SEQ ID nr.1 and nr.6 in different concentrations (for ex. 400 nm, 800 nm, 1600 nM) and as described below. For comparison purposes, the solutions were also prepared with alternative concentration of the probe for allele “wild-type” (e.g., 250 nM, 500 nM).

I—Compositions for Mutation Detection Located at Bases -124 Upstream of the Initiation Codon ATG of the hTERT Gene

Inventive Composition Ia:
Solution reagent for Real-Time PCR “TaqMan® Universal PCR Master Mix” at a concentration 1×;
Oligonucleotide with the sequence: SEQ ID n. 1, at a concentration of 900 nM;
Oligonucleotide with the sequence: SEQ ID n. 2, at a concentration of 900 nM;
Oligonucleotide probe with the sequence: SEQ ID n. 3, at a concentration of 250 nM, containing modifications such as the incorporation of the compound known as YAKIMA YELLOW® and tetra-methyl-rhodamine (TAMRA) in the 5′ and 3′ terminals, respectively;
Oligonucleotide probe with the sequence: SEQ ID n. 4, at a concentration of 1600 nM, containing modifications such as the incorporation of the compound known as 6-carboxi-fluorescein (6-FAM) and tetra-methyl-rhodamine (TAMRA) in the 5′ and 3′ terminals, respectively;
Water bi-distilled and bi-deionized.

Inventive Composition Ib:

This composition is prepared similarly to composition Ia but being the oligonucleotide probe concentration replaced with the one identified as: SEQ ID NO. 4, in a concentration of 800 nm.

Inventive Composition Ic:

This composition is prepared similarly to composition Ia but being the oligonucleotide probe concentration replaced with the one identified as: SEQ ID NO. 4, in a concentration of 400 nm.

Comparative Composition Id:

This composition is prepared similarly to composition Ia but being the oligonucleotide probe with the sequence: SEQ ID NO. 3, at a concentration of 500 nM.

II—Compositions for Mutation Detection Located at -146 Bases Upstream of the Initiation Codon ATG of the hTERT Gene

Inventive Composition IIa:
Solution reagent for Real-Time PCR “TaqMan® Universal PCR Master Mix” at a concentration 1×;
Oligonucleotide with the sequence: SEQ ID n. 1, at a concentration of 900 nM;
Oligonucleotide with the sequence: SEQ ID n. 2, at a concentration of 900 nM;
Oligonucleotide probe with the sequence: SEQ ID n. 5, at a concentration of 250 nM, containing modifications in the 5′ and 3′ terminals, respectively;
Oligonucleotide probe with the sequence: SEQ ID n. 6, at a concentration of 1600 nM, containing modifications in the 5′ and 3′ terminals, respectively;
Water bi-distilled and bi-deionized.

The mentioned modifications at the 5′ and 3′ terminals sequences of the probes correspond to the addition of fluorophores (FAM, Yakima yellow) and quencher (TAMRA), etc. known to the expert.

Inventive Composition IIb:

This composition is prepared similarly to composition IIa however the oligonucleotide probe with the sequence: SEQ ID NO. 6, is present at a concentration of 800 nM.

Inventive Composition IIc:

This composition is prepared similarly to composition IIa however the oligonucleotide probe with the sequence: SEQ ID NO. 6, is present at a concentration of 400 nM.

Comparative Composition IId:

This composition is prepared similarly to composition Ia however the oligonucleotide probe with the sequence: SEQ ID NO. 5, is present at a concentration of 500 nM.

III Compositions for Simultaneous Detection of the Mutations Located at -124 and -146 Bases Upstream of the Initiation Codon ATG of the hTERT Gene:

Inventive Composition IIIa:
Solution reagent for Real-Time PCR “TaqMan® Universal PCR Master Mix” at a concentration 1×;
Oligonucleotide with the sequence: SEQ ID n. 1, at a final concentration of 900 nM;
Oligonucleotide with the sequence: SEQ ID n. 2, at a final concentration of 900 nM;
Oligonucleotide with the sequence: SEQ ID n. 3, at a final concentration of 900 nM;
Oligonucleotide probe with the sequence: SEQ ID n. 4, at a concentration of 1600 nM, containing modifications in the 5′ and 3′ terminals, respectively;
Oligonucleotide probe with the sequence: SEQ ID n. 5, at a concentration of 250 nM, containing modifications in the 5′ and 3′ terminals, respectively;
Oligonucleotide probe with the sequence: SEQ ID n. 6, at a concentration of 1600 nM, containing modifications in the 5′ and 3′ terminals, respectively
Water bi-distilled and bi-deionized.

Inventive Composition IIIb:

This composition is prepared similarly to composition IIIa, however the oligonucleotide probe with the sequence: SEQ ID NO. 4, is present in a concentration of 800 nM.

Inventive Composition IIIc:

This composition is prepared similarly to composition IIIa, however the oligonucleotide probe with the sequence: SEQ ID NO. 4, is present in a concentration of 400 nM.

Inventive Composition IIId:

This composition is prepared similarly to composition IIIa, however the oligonucleotide probe with the sequence: SEQ ID NO. 6, is present at a concentration of 800 nM.

Inventive Composition IIIe:

This composition is prepared similarly to composition IIIa, however the oligonucleotide probe with the sequence: SEQ ID NO. 6, is present at a concentration of 400 nM.

Comparative Composition IIIf:

This composition is prepared similarly to composition Ia, however the oligonucleotide probe with the sequence: SEQ ID NO. 3, is present at a concentration of 500 nM.

Comparative Composition IIIg:

This composition is prepared similarly to composition but being Ia probe oligonucleotide with the sequence: SEQ ID NO. 5, present at a concentration of 500 nM.

### Example 3—Preparation of Compositions for Detecting Mutations

The compositions of the reaction mixtures for the detection of mutations c.-124 C>T and c.-146 C>T in the promoter of the gene hTERT were prepared with the addition of DNA from a biological test sample, approximately 100 ng of DNA, (it can be used lesser quantity, ranging from 1 ng to 500 ng) to the PCR compositions mentioned above, with the concentration of the probes with SEQ ID nr.3 and SEQ ID nr.6, adjusted to values ranging from 400 to 1600 nM, like described in the previous example. The compositions for detection were thus prepared with DNA samples containing the c.124 C>T mutation or samples containing the c.146 C>T mutation “serial diluted” in “wild-type” DNA samples.

Likewise, compositions for detection were prepared for comparative purposes, through the addition of DNA from the biological test sample to comparative solutions from the previous example.

### Example 4—Method for the Detection of the Mutations c.-124 C>T and c.-146 C>T Located in the Promoter of the hTERT Gene

For carrying out the method of the present invention there were prepared different compositions for detecting mutations as described in the previous example.

In this example it was used DNA from cell lines or from DNA tumor tissue, either fresh or Formalin Fixed Paraffin Embedded.

The compositions for detection from the previous example were subjected to amplification and hybridization processes in Real-Time PCR and Digital PCR. The compositions were also subjected to simultaneous assays (Multiplex) using in the same reaction probes for both mutations in analysis, being the probes labeled with different fluorophores. Among the most common fluorophores are known compounds known by Alexa Fluor FAM, TET, JOE, VIC, HEX, Cy3, ATTO 550, TAMRA, ROX, Cy5, Cy5.5.

For carrying out the method in Real Time PCR the ABI PRISM® 7500 Fast machine marketed by Scientific ThermoFischer was used.

Amplification and hybridization conditions:
1^{st} stage: 1 cycle of 10 minutes at 95° C.
2^{nd} stage: 45 cycles, comprising 30 seconds at 92° C., 1 minute at 60° C., with temperature drop of 0.2° C. per cycle starting from cycle 25
3^{rd} stage: 1 cycle of 1 minute at 57° C., with signal acquisition

### Example 5—Method for the Detection of the Mutations c.-124 C>T and c.-146 C>T Located in the Promoter of the hTERT Gene in a Urine Sample

Patient urine samples were obtained from bladder cancer patients in clinical or monitoring programs (cystoscopy) for tumor recurrence prevention. The urine was centrifuged at a force exceeding 1000 times the gravitational force (1000G) for a time period exceeding 5 minutes to pellet the epithelial cells from the bladder present in the urine. DNA was extracted from the obtained pelleted cells using common methods known in the art such as QIAamp® commercialized by Qiagen company or similar methods. Between 1 and 500 ng of the DNA obtained was added to the compositions for amplification and detection of mutations prepared as described in Examples 1, 2 and 3.

The compositions for detection from the example 3 were subjected to amplification and hybridization processes in Real-Time PCR and Digital PCR. The compositions were also subjected to simultaneous assays (Multiplex) using in the same reaction probes for both mutations in analysis, being the probes labeled with different fluorophores. Among the most common fluorophores are known compounds known by Alexa Fluor FAM, TET, JOE, VIC, HEX, Cy3, ATTO 550, TAMRA, ROX, Cy5, Cy5.5.

For carrying out the method in Real Time PCR the ABI PRISM® 7500 Fast machine marketed by Scientific ThermoFischer was used.

Amplification and Hybridization Conditions:
1^{st} stage: 1 cycle of 10 minutes at 95° C.
2^{nd} stage: 45 cycles, comprising 30 seconds at 92° C., 1 minute at 60° C., with temperature drop of 0.2° C. per cycle starting from cycle 25
3^{rd} stage: 1 cycle of 1 minute at 57° C., with signal acquisition

Using this method, the changes c.-124 C>T and c.-146 C>T in the hTERT gene promoter was detected in patients with bladder cancer both with low or high-grade, as well as in patients with recurrent bladder cancer (FIG. 7).

In Conclusion:

The specificity of the probes was verified, i.e. if was evaluated if the probes for c.-124 C>T mutation (probes with SEQ ID Nr.3 and SEQ ID nr.4) did not produce amplification of a heterozygous DNA sample for the c.-146 C>T alteration (DNA MUT 146) and vice versa (with the probes with SEQ ID nr.5 and SEQ ID nr.6) (FIGS. 3A and 3B). It was then verified that there are no phenomena of cross detection or false detection between the referred probes and the respective mutations.

In DNA samples derived from biological fluids, in which there are small amounts of mutated alleles in an environment dominated by “wild-type” alleles, the analytical sensitivity of -124T and -146T probes at a final concentration of 400 nm at its respective mutation detection capability is maintained up to a maximum of 3.125% in the case of probe -124T and 6, 25% for the -146T probe (FIGS. 4a and b, respectively), which clearly shows the high efficacy of probes for the detection of mutations in high “wild-type” allele dilution conditions and therefore the ultra-sensitivity of the method of the present invention.

Under conditions that used 25% ADNMUT/ADNWT, it can be observed that in low concentration of the probe for the allele “wild-type” (e.g. 250 nM) the use of a higher concentration of the probe for the mutated alleles -124T and -146T (400-800-1600 nM) results in a significant increase of the detection signal of the mutated alleles (FIG. 5 panels a and B, respectively), which clearly indicates that by manipulating the concentration of the probes it is possible to adjust the sensitivity of the method of the present invention, for detecting the mutations in question.

To the increase the efficiency of detection of the mutated alleles -124T and -146T, it corresponded a significant increase in the sensitivity of the analytical method of the invention, passing to minimum values of detection of 1.56% in the case of -124T allele and 0.78% in the case of -146T allele (FIGS. 6A and B, respectively), which clearly indicates that by manipulating the concentration of the probes is possible to enhance the analytical sensitivity of the invention method for detecting the mutations in question, maintaining its specificity.

SEQUENCE LISTING

I-Primers
SEQ ID nr. 1:
5′-CCACGTGCGCAGCAGGAC-3′
SEQ ID nr. 2:
5′-CCGTCCTGCCCCTTCACCTT-3′
II-Specific probes for the c.-124 C > T
mutations in the promoter of the hTERT gene
SEQ ID nr. 3:
Yakima Yellow Dye-5′-AGGGCCCGGAGGGGGCT-3′-TAMRA
SEQ ID nr. 4:
FAM-5′-AGGGCCCGGAAGGGGCT-3′-TAMRA
III-Specific Probes for the c.-146 C > T
mutations in the promoter of the hTERT gene
SEQ ID nr. 5:
Yakima Yellow Dye-5′-CGGGGACCCGGGAGGGGT-3′-TAMRA
SEQ ID nr. 6:
FAM-5′-CGGGGACCCGGAAGGGGT-3′-TAMRA

### REFERENCES

1. Hayflick, L. and P. S. Moorhead, The serial cultivation of human diploid cell strains. Exp Cell Res, 1961. 25: p. 585-621.
2. Blackburn, E. H., Structure and function of telomeres. Nature, 1991. 350(6319): p. 569-73.
3. Greider, C. W., Telomerase is processive. Mol Cell Biol, 1991. 11(9): p. 4572-80.
4. Szostak, J. W. and E. H. Blackburn, Cloning yeast telomeres on linear plasmid vectors. Cell, 1982. 29(1): p. 245-55.
5. Gunes, C. and K. L. Rudolph, The role of telomeres in stem cells and cancer. Cell, 2013. 152(3):p.390-3
6. Murnane, J. P., Telomere dysfunction and chromosome instability. Mutat Res, 2012. 730(1-2): p. 28-36.
7. Cesare, A. J. and R. R. Reddel, Alternative lengthening of telomeres: models, mechanisms and implications. Nat Rev Genet, 2010. 11(5): p. 319-30.
8. Kim, N. W., et al., Specific association of human telomerase activity with immortal cells and cancer. Science, 1994. 266(5193): p. 2011-5.
9. Kyo, S., et al., Understanding and exploiting hTERT promoter regulation for diagnosis and treatment of human cancers. Cancer Sci, 2008. 99(8): p. 1528-38.
10. Aubert, G. and P. M. Lansdorp, Telomeres and aging. Physiol Rev, 2008. 88(2): p. 557-79.
11. Killela, P. J., et al., TERT promoter mutations occur frequently in gliomas and a subset of tumors derived from cells with low rates of self-renewal. Proc Natl Acad Sci USA, 2013. 110(15): p. 6021-6.
12. Liu, X., et al., Highly prevalent TERT promoter mutations in aggressive thyroid cancers. Endocr Relat Cancer, 2013. 20(4): p. 603-10.
13. Nault, J. C., et al., High frequency of telomerase reverse-transcriptase promoter somatic mutations in hepatocellular carcinoma and preneoplastic lesions. Nat Commun, 2013. 4: p. 2218.
14. Vinagre, J., et al., Frequency of TERT promoter mutations in human cancers. Nat Commun, 2013. 4: p. 2185.
Lisbon, 2 Jun. 2016

1. A set of nucleotide sequences for the detection of mutations c.-124 C>T and/or c.-146 C>T in the promoter of the gene hTERT characterized by comprising SEQ ID Nr. 1 to SEQ ID Nr.2 and at least SEQ ID Nr.3 to SEQ ID nr.4 and/or SEQ ID nr.5 SEQ ID nr.6.

2. A composition for amplification and hybridization by PCR for the detection of mutations C.-124 C>T and/or c.-146 C>T in the promoter of the gene hTERT characterized by comprising, in addition to reagent solutions for PCR, the nucleotide sequences SEQ ID nr.1 to SEQ ID Nr.2 and at least SEQ ID Nr.3 to SEQ ID nr.4 and/or SEQ ID nr.5 to SEQ ID nr.6 as described in claim 1.

3. A composition, according to claim 1, characterized by at least one of SEQ ID Nr.3 to SEQ ID nr.6 to be present in this composition in a final concentration of 400 nm to 1600 nM.

4. A composition, according to claim 1, characterized by at least one of SEQ ID Nr.3 to SEQ ID nr.6 nucleotide sequences to be present in this composition in a final concentration of 800 nm to 1600 nM.

5. A composition, according to claim 1, characterized by the SEQ ID Nr.3 and SEQ ID nr.5 nucleotide sequences to be present in this composition in the final concentration of 250 nm to 500 nM and by the SEQ ID Nr.4 and SEQ ID nr.6 nucleotide sequences to be present in this composition in a final concentration of 800 nm to 1600 nM.

6. A composition, according to claim 1, characterized by comprising the following concentrations of SEQ ID Nr.3 SEQ ID nr.6:
i) 250 nM to 500 nM of SEQ ID Nr.3, 400 nm to 1600 nM of SEQ ID nr.4, 250 nM to 500 nM of SEQ ID nr.5 and 400 nm to 1600 nM of SEQ ID nr.6,
or
ii) 250 nM to 500 nM of SEQ ID Nr.3, 800 nm to 1600 nM of SEQ ID nr.4, 250 nM to 500 nM of SEQ ID nr.5 and 800 nm to 1600 nM of SEQ ID nr.6.

7. A composition for the detection of mutations c.-124 C>T and c.-146 C>T in the promoter of the gene hTERT characterized by comprising, in addition to a human DNA obtained from a biological sample in vitro, at least one composition for PCR amplification and hybridization, as written in claim 2.

8. A composition, according to claim 7, characterized by comprising an amount of DNA of the testing sample, in the referred, composition at a final concentration of less than 500 ng, less than 100 ng, less than 50 ng and more than 1 ng.

9. A kit for the detection of mutations c.-124 C>T and/or C.-146 C>T in the promoter of the gene hTERT comprising at least one composition for the detection of mutations as described in claim 7.

10. A kit, according to claim 9, characterized by further comprising at least one DNA sample containing the mutation c.-124 C>T and/or c.-146 C>T in the promoter of the gene hTERT.

11. An in vitro method for the detection of the mutations c.-124 C>T and/or c.-146 C>T in the promoter of the gene hTERT characterized by comprising the following steps:
a) Preparation of composition for detection as described in claim 7, by the promotion of a contact of the DNA extracted from a biological sample, to be tested for referred mutation, with the composition for amplification and hybridization by PCR,
b) Amplification and hybridization of the DNA sample of the composition detection by PCR reaction,
c) Analysis of the amplification curves obtained in (b) and verification of the presence of exponential amplification of a fluorescence signal, which corresponds to the positivity for the presence of the c.-124 C>T mutation and/or c.-146 C>T mutation in the promoter of the gene hTERT promoter in the sample under analysis.

12. A method, according to claim 11, where the biological sample of (a) is derived from urine, plasma, cerebrospinal fluid, aspiration cytology of a human being.

13. A method, according to claim 12, where the amount of DNA in the biological sample composition (a) varies at a final concentration of less than 500 ng, less than 100 ng, less than 50 ng and more than 1 ng.

14. A method, according to claim 11, where the amplification conditions are as follows:
1^{st} stage: 1 cycle of 10 minutes at 95° C.
2^{nd} stage: 45 cycles, comprising 30 seconds at 92° C., 1 minute at 60° C., with temperature drop of 0.2° C. per cycle starting from cycle 25
3^{rd} stage: 1 cycle of 1 minute at 57° C., with signal acquisition.

15. A method for treatment of human diseases associated with the mutation c.124 C>T and/or c.-146 C>T in the promoter of the gene hTERT characterized by comprising the administration of a suitable amount of an inhibitor of the promoter of the gene hTERT to an individual whose biological sample indicated positive in a detection test for the mutations c.-124 C>T and/or c.-146 C>T in the promoter of the gene hTERT, which comprises the steps of a method as described in claim 11.
